(19) United States
(12) Patent Application Publication
(10) Pub. No.: US 2022/0396790 A1
(43) Pub. Date: Dec. 15, 2022

(54) HIGH-THROUGHPUT SCREENING PLATFORM FOR ENGINEERING NEXT-GENERATION GENE THERAPY VECTORS

(71) Applicant: Duke University, Durham, NC (US)

(72) Inventors: Charles A. Gersbach, Chapel Hill, NC (US); Matthew Gemberling, Durham, NC (US); Tyler Klann, Durham, NC (US)

(21) Appl. No.: 17/633,467

(22) PCT Filed: Aug. 8, 2020
(86) PCT No.: PCT/US2020/045544

§ 371 Date: Feb. 7, 2022

Related U.S. Application Data
(60) Provisional application No. 62/884,536, filed on Aug. 8, 2019.

(51) Int. Cl. C12N 15/10; C12N 15/86

(52) U.S. Cl. CPC C12N 15/1086; C12N 15/86; C12N 15/1051; C12N 15/1065; C12N 15/1075; C12N 2750/14143; C12N 2740/16043; C12Q 2563/107; C12Q 2563/179

(57) ABSTRACT

Disclosed herein are methods of identifying or engineering a polynucleotide sequence for directing tissue-specific gene expression. The methods may further include creating a regulatory element fragment library. Further disclosed are vectors comprising a tissue-specific regulatory element identified by the methods.

### CROSS-REFERENCE TO RELATED APPLICATIONS

This application claims priority to U.S. Provisional Patent Application No. 62/884,536, filed Aug. 8, 2019, which is incorporated herein by reference in its entirety.

### FIELD

This disclosure relates to compositions and methods for identifying or engineering polynucleotides that direct tissue-specific or cell-specific gene expression and vectors comprising the same.

### INTRODUCTION

The ability to target a specific tissue or cell type within a tissue is of great interest when developing gene therapies. There are many cases in which expression of a protein is therapeutic in one cell type but can be toxic in another. While many cell type-specific promoters have been reported, they are typically inadequate for clinical gene therapy. For example, most cell type- or tissue-specific promoters are weaker than the constitutive promoters typically used in preclinical studies. Additionally, most of these promoters may be selective for certain types of tissues, but not specific for a particular tissue. For example, any muscle-specific promoter typically has activity in skeletal, cardiac, and smooth muscle, making it difficult to target only one of these tissues specifically. These limitations have manifested in preclinical development and clinical trials of particular gene therapies. For example, delivery of the gene encoding Calpain3 to skeletal muscle is able to reverse pathology associated with Limb-Girdle Muscular Dystrophy 2A (LGMD2A). However, expression of Calpain3 in cardiac muscle is toxic and can cause cardiac lesions (Roudaut, C. et al. Circulation 2013, 128, 1094-1104). Like LGMD2A, X-Linked Myotubular Myopathy (XMTM) can be treated by the delivery and expression of the gene encoding MTM1 in skeletal muscle, however animal studies have shown the potential for cardiac fibrosis and lesions to appear if MTM1 is expressed in the heart (Childers, M. K. et al. Sci. Transl. Med. 2014, 6, 220ra210). Common among these two diseases is the need to restrict expression of the therapeutic protein to skeletal muscle. As AAV8 and AAV9 transduce both skeletal and cardiac muscle with high efficiency, one method for restricting expression lies within the gene therapy vector promoter. The current status of promoter element design typically encompasses taking the promoter elements from a gene known to be expressed in a tissue of interested and selecting a fragment that drives a desired expression pattern. While this strategy can be effective in some cases, it limits the pool of potential regulatory elements and may not be applicable to all cell types. There is a need for technologies that express genes specifically in any cell or tissue type, in multiple desired tissues, and/or in a regulatable fashion in response to pharmacologic treatment, while preventing expression in off-target tissues.

### SUMMARY

In an aspect, the disclosure relates to methods of identifying a promoter sequence for directing tissue-specific or cell-specific gene expression. The method may include identifying one or more DNA fragments from a first cell type, wherein each DNA fragment is 50 nt to 200 nt in length and present in a genomic region comprising at least one epigenetic feature in the first cell type, wherein the epigenetic feature is selected from open chromatin and histone mark and DNA methylation, and wherein the epigenetic feature is not present in the same genomic region in at least one second cell type; inserting into a vector at least one promoter sequence comprising at least one of the one or more DNA fragments and at least one sequence tag, wherein the sequence tag comprises a polynucleotide sequence that is specific for each DNA fragment; transducing one or more vectors into an expression cell; and determining the level of transcription of the sequence tag in the expression cell, wherein an increased level of transcription of the sequence tag in the expression cell relative to a control indicates that the promoter sequence directs tissue-specific or cell-specific gene expression.

In some embodiments, the vector further includes a polynucleotide encoding a reporter downstream of and operably linked to the at least one promoter sequence. In some embodiments, the reporter comprises a fluorescent protein. In some embodiments, the method further includes sequencing the one or more DNA fragments prior to inserting into a vector the at least one promoter sequence. In some embodiments, the method further includes synthesizing the one or more DNA fragments prior to inserting into a vector the at least one promoter sequence. In some embodiments, the epigenetic feature is not present in the same genomic region in at least two second cell types. In some embodiments, the epigenetic feature is not present in the same genomic region in at least one second cell type but is present in the same genomic region in at least one third cell type. In some embodiments, the first cell type and the second cell type are from different tissues. In some embodiments, the first cell type and the second cell type are different cell types. In some embodiments, the vector comprises a combination of at least about 2, at least about 3, or at least about 4 of the one or more DNA fragments. In some embodiments, the promoter sequence comprises a combination of at least about 2, at least about 3, or at least about 4 of the one or more DNA fragments. In some embodiments, the one or more DNA fragments are identified by comparing DNAse hypersensitivity data for the first cell type to DNAse hypersensitivity data for the second cell type. In some embodiments, the DNAse hypersensitivity data is obtained by DNAse-seq or ATAC-seq. In some embodiments, the one or more DNA fragments are identified by comparing histone modification data for the first cell type to histone modification data for the second cell type. In some embodiments, the histone modification data is obtained by ChIP-seq. In some embodiments, the level of transcription of the sequence tag in the expression cell is determined by quantitative DNA sequencing. In some embodiments, the method further includes comparing the level of transcription of the sequence tag in the expression cell to the level of transcription of a different sequence tag corresponding to a different promoter sequence. In some embodiments, the method further includes comparing the level of transcription of the sequence tag in the expression cell to the level of transcription of the same sequence tag in a different cell type. In some embodiments, the vector is a lentiviral or adeno-associated viral (AAV) vector. In some embodiments, the first cell type is a cardiac cell, skeletal muscle cell, smooth muscle cell, endothelial cell, intestinal cell, epithelial cell, liver cell, retinal cell, hematopoietic stem cell, satellite cell, CNS cell, astrocyte, glial cell, brain cell, neuronal cell, or neuronal subtype cell. In some embodiments, the neuronal subtype cell is a dopaminergic neuron, gabaergic neuron, or glutamatergic neuron.

In a further aspect, the disclosure relates to a vector comprising the tissue-specific or cell-specific promoter sequence identified by the method as detailed herein. In some embodiments, the tissue-specific or cell-specific promoter sequence is a cardiac muscle-specific promoter or a skeletal muscle-specific promoter.

The disclosure provides for other aspects and embodiments that will be apparent in light of the following detailed description and accompanying figures.

### BRIEF DESCRIPTION OF THE DRAWINGS

FIG. 1 is an overview of the platform for gene therapy vectors. Current viral vectors are taken up by multiple tissues. Combinatorial assembly of natural epigenetic control elements and high-throughput in vivo screening are used to engineer gene regulatory cassettes that are specific to certain tissues, allowing for safer and more effective gene expression with reduced off-target toxicities.

FIG. 2 are images showing Dystrophin staining of skeletal and heart tissue in the CRISPR/Cas9-treated mdx mouse model of Duchenne muscular dystrophy.

FIG. 3 is a schematic overview of the generation of regulatory element fragment libraries. DNase-seq or ATAC-seq provides regions of the genome that are characterized by open chromatin (˜100,000 regions/tissue) in particular tissues. Those regions can be identified and synthesized in a high-throughput manner for incorporation into AAV vectors.

FIG. 4 is a schematic diagram of the pipeline for the identification of novel promoter elements.

FIGS. 5A-5C. FIG. 5A is a graph showing the distribution of the DNA fragments in the skeletal muscle library, confirming at least 99.9% inclusion of the 99,791 DNA fragments. FIG. 5B is a graph showing the distribution of the DNA fragments in the cardiac muscle library, confirming at least 99.9% inclusion of the 149,934 DNA fragments. FIG. 5C shows sequencing results of the pool of vectors around the barcode region of the vector, indicating a normal distribution of library members in the pool.

FIGS. 6A-6I. FIG. 6A is a schematic diagram for the transfection of human cells with the skeletal or cardiac muscle library. FIGS. 6B-6E are graphs showing the expression of GFP in AC16 cardiomyocytes transfected with the cardiac library (FIG. 6D) or skeletal muscle library (FIG. 6C), or a negative control (FIG. 6B), with a comparison in FIG. 6E. Graphs are shown for the comparison of all the DNA fragments (barcodes) versus those that affected GFP expression in cardiomyocytes transduced with the cardiac muscle library (FIG. 6F), in skeletal myoblasts transduced with the cardiac muscle library (FIG. 6G), in skeletal myoblasts transduced with the skeletal muscle library (FIG. 6H), and in cardiomyocytes transduced with the skeletal muscle library (FIG. 6I).

FIGS. 7A-7E. FIG. 7A is a schematic diagram for the transfection of mice with the skeletal muscle library or cardiac muscle library. Graphs show the comparison of all the DNA fragments (barcodes) versus those that affected GFP expression in skeletal or cardiac cells with the skeletal library (FIG. 7B) and in skeletal or cardiac cells with the cardiac library (FIG. 7C). The distribution of DNA fragments affecting GFP expression among various tissues is shown for the skeletal library (FIG. 7D) and the cardiac library (FIG. 7E).

### DETAILED DESCRIPTION

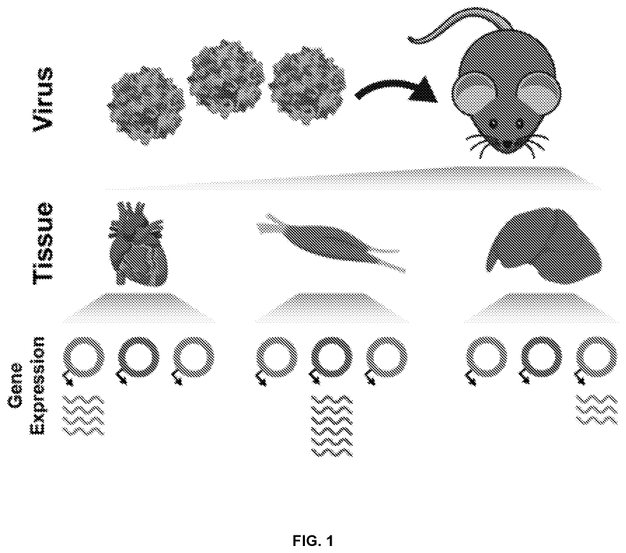

Detailed herein is a high-throughput, unbiased method to identify novel promoter elements and sequences that can be used to express the viral cargo in a tissue- or cell type-specific manner (FIG. 1). The platform leverages expertise in natural human epigenetics, high-throughput genomics, next-generation DNA sequencing, viral vector engineering, and gene therapy. Use of this method has enabled the identification of new regulatory modules that can drive gene expression in, for example, cardiac or skeletal muscle in vivo. The compositions and methods detailed herein may be used to identify sequences directing tissue-specific or cell-specific gene expression, such as expression specific for a single tissue or cell type but not others, or expression specific for multiple tissue or cell types but not others.

Gene therapy vectors are typically utilized to perform gene transfer or deliver genome editing tools for gene correction strategies. These strategies rely on the production of one or more proteins expressed from the packaged vectors. Firstly, these vectors are delivered to the cell types of interest in order to achieve therapeutic effect. The platform for engineering novel gene regulatory systems can be used with any method for delivering transgenes or genome editing tools, including, for example, AAV delivery. AAV can efficiently transduce a wide variety of cell types, has a low rate of genome integration, and can be maintained as an episome, which may allow for years of stable expression. AAV has numerous serotypes with tropisms for different tissues in vivo. In addition to naturally occurring serotypes, engineering of the viral capsids can be utilized to restrict the ability of the virus to transduce specific cells types. While serotype selection can be used to restrict the ability of AAV to transduce some cell types, there are no natural serotypes with perfect selectivity for a single tissue. For example, serotypes that can transduce skeletal muscle typically also transduce cardiac muscle, and vice versa. Moreover, all serotypes transduce hepatocytes in the liver to some extent, as this organ is responsible for filtering the blood. As an additional layer of regulation, gene expression can also be controlled after cell entry via transcriptional modulation. The gene therapy vectors with promoters identified by the methods detailed herein may be used to tightly control expression after viral entry. This approach will also allow for the use of more common and well-studied serotypes of AAV that may have less selective tropisms.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a target region that is recognized and bound by the CRISPR/Cas-based gene editing system.

As used herein, the term “cloning” refers to the process of ligating a polynucleotide into a vector and transferring it into an appropriate host cell for duplication during propagation of the host.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The term “host cell” is a cell that is susceptible to transformation, transfection, transduction, conjugation, and the like with a polynucleotide construct or expression vector. Host cells can be derived from plants, bacteria, yeast, fungi, insects, animals, protozoans, etc.

As used herein, the term “gene” means the polynucleotide sequence comprising the coding region of a gene, e.g., a structural gene, and the including sequences located adjacent to the coding region on both the 5′ and 3′ ends for a distance of about 1 kb on either end such that the gene corresponds to the length of the full-length mRNA. The sequences which are located 5′ or upstream of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA, for example, heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript) or untranslated regions (UTRs). UTRs do not form the protein-coding region of the gene. The 5′ flanking region may contain regulatory sequences such as promoters and enhancers which control or influence the transcription of the gene. The 3′ flanking region may contain sequences which direct the termination of transcription, post-transcriptional cleavage and polyadenylation.

“Gene expression” describes the conversion of the DNA gene sequence information into transcribed RNA (the initial unspliced RNA transcript or the mature mRNA) or the encoded protein product. The expression level of a gene may refer to an amount or a concentration of a transcription product, such as mRNA, or of a translation product, such as a protein or polypeptide. Gene expression can be monitored by, for example, measuring the levels of either the entire RNA or protein products of the gene or their subsequences.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity or for a gene expression level may be defined in accordance with standard practice. A control may be a subject, or a sample therefrom, whose disease state is known. The subject, or sample therefrom, may be at any stage of disease. The subject, or sample therefrom, may be healthy, diseased, diseased prior to treatment, diseased during treatment, or diseased after treatment, or a combination thereof.

“Nucleic acid” or “oligonucleotide” or“polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand may provide a probe that may hybridize to a target sequence under stringent hybridization conditions. Thus, a polynucleotide also encompasses a probe that hybridizes under stringent hybridization conditions. Polynucleotides can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

Polynucleotides are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a polynucleotide sequence, even if internal to a larger oligonucleotide, also may be said to have 5′ and 3′ ends. In either a linear or circular polynucleotide, discrete elements are referred to as being “upstream” or 5′ of the “downstream” or 3′ elements. This terminology reflects the fact that transcription proceeds in a 5′ to 3′ fashion along the polynucleotide strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, an oligonucleotide or polynucleotide “having a nucleotide sequence encoding a gene” means a polynucleotide sequence comprising the coding region of a gene, or in other words, the nucleic acid sequence which encodes a gene product. The coding region may be present in either a cDNA, genomic DNA, or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the vector may contain endogenous enhancers/promoters, splice junctions, intervening sequences, polyadenylation signals, etc., or a combination of both endogenous and exogenous control elements.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Recombinant” when used with reference, e.g., to a cell, or polynucleotide, protein, or vector, indicates that the cell, nucleic acid, protein, or vector, has been modified by the introduction of a heterologous nucleic acid or protein or the alteration of a native polynucleotide or protein, or that the cell is derived from a cell so modified. Thus, for example, recombinant cells express genes that are not found within the native (non-recombinant) form of the cell or express native genes that are otherwise abnormally expressed, under expressed, or not expressed at all. For example, the term “recombinant DNA molecule” as used herein refers to a DNA molecule which is comprised of segments of DNA joined together by means of molecular biological techniques. The term “recombinant protein” or “recombinant polypeptide” as used herein refers to a protein molecule which is expressed from a recombinant DNA molecule or recombinant polynucleotide.

An “open reading frame” includes at least 3 consecutive codons which are not stop codons. The term “codon” as used herein refers to any group of three consecutive nucleotide bases in a given messenger RNA molecule, or coding strand of DNA or polynucleotide that specifies a particular amino acid, a starting signal, or a stopping signal for translation. The term codon also refers to base triplets in a DNA strand.

The terms “in operable combination,” “in operable order,” and “operably linked” as used herein refer to the linkage of polynucleotide sequences in such a manner that a polynucleotide molecule capable of directing the transcription of a given gene and/or the synthesis of a desired protein molecule is produced. The term also refers to the linkage of amino acid sequences in such a manner so that a functional protein is produced.

As used herein, the term “restriction endonuclease” or “restriction enzyme” refers to a member or members of a classification of catalytic molecules that bind a cognate sequence of a polynucleotide and cleave the polynucleotide at a precise location within that sequence. Restriction endonuclease may be bacterial enzymes. Restriction endonuclease may cut double-stranded DNA at or near a specific nucleotide sequence.

As used herein, “recognition site” or “restriction site” refers to a sequence of specific bases or nucleotides that is recognized by a restriction enzyme if the sequence is present in double-stranded DNA; or, if the sequence is present in single-stranded RNA, the sequence of specific bases or nucleotides that would be recognized by a restriction enzyme if the RNA was reverse transcribed into cDNA and the cDNA employed as a template with a DNA polymerase to generate a double-stranded DNA; or, if the sequence is present in single-stranded DNA, the sequence of specific bases or nucleotides that would be recognized by a restriction enzyme if the single-stranded DNA was employed as a template with a DNA polymerase to generate a double-stranded DNA; or, if the sequence is present in double-stranded RNA, the sequence of specific bases or nucleotides that would be recognized by a restriction enzyme if either strand of RNA was reverse transcribed into cDNA and the cDNA employed as a template with a DNA polymerase to generate a double-stranded DNA. The term “unique restriction enzyme site” or “unique recognition site” indicates that the recognition sequence for a given restriction enzyme appears once within a polynucleotide.

As used herein, the term “regulatory element” refers to a genetic element which controls some aspect of the expression of polynucleotide sequences. For example, a promoter is a regulatory element that facilitates the initiation of transcription of an operably linked coding region. Other regulatory elements may include splicing signals. polyadenylation signals, termination signals, and the like. Transcriptional control signals in eukaryotes include “promoter” and “enhancer” elements. Promoters and enhancers include short arrays of polynucleotide sequences that interact specifically with cellular proteins and RNAs involved in transcription (Maniatis et al. Science 1987, 236, 1237), incorporated herein by reference). A promoter may be “inducible”, initiating transcription in response to an inducing agent or, in contrast, a promoter may be “constitutive”, whereby an inducing agent does not regulate the rate of transcription. A promoter may be regulatable. For example, a regulatable promoter may include an inducible promoter. A promoter may direct tissue-specific expression of a gene. Conventional promoter and enhancer elements have been isolated from a variety of eukaryotic sources such as, for example, genes in yeast, insect and mammalian cells, and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss et al., Trends Biochem. Sci. 1986, 11, 287; and Maniatis et al., supra (1987)). For example, the SV40 early gene enhancer is very active in a wide variety of cell types from many mammalian species and has been widely used for the expression of proteins in mammalian cells (Dijkema et al. EMBO J. 1985, 4, 761). Two other examples of promoter/enhancer elements active in a broad range of mammalian cell types are those from the human elongation factor 10 gene (Uetsuki et al. J. Biol. Chem. 1989, 264, 5791; Kim et al. Gene, 1990, 91, 217; Mizushima et al. Nuc. Acids. Res. 1990, 18, 5322) and the long terminal repeats of the Rous sarcoma virus (Gorman et al. Proc. Natl. Acad. Sci. USA 1982, 79, 6777) and the human cytomegalovirus (Boshart et al. Cell 1985, 41, 521).

As used herein, the term “promoter/enhancer” denotes a segment of a polynucleotide that contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

“Replication origins” are unique polynucleotide segments that contain multiple short repeated sequences that are recognized by multimeric origin-binding proteins and which play a key role in assembling DNA replication enzymes at the origin site.

“Sample” or “test sample” as used herein can mean any sample in which the presence and/or level of a target or gene is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Specific expression” is the expression of a gene product that is limited to one or a few tissues or cells (spatial limitation) and/or to one or a few developmental stages (temporal limitation). For example, gene expression may occur in one tissue or cell type, but expression of the same gene may be reduced to 0, less than 1%, less than 2%, less than 3%, less than 4%, less than 5%, less than 10%, less than 15%, less than 20%, or less than 25% of the expression level in a second tissue or cell type. Expression may be controlled by a drug, such as wherein gene expression occurs upon administration of a drug but gene expression is reduced to 0, less than 1%, less than 2%, less than 3%, less than 4%, less than 5%, less than 10%, less than 15%, less than 20%, or less than 25% when the drug is not administered. Conversely, gene expression may occur in the absence of a drug but gene expression is reduced to 0, less than 1%, less than 2%, less than 3%, less than 4%, less than 5%, less than 10%, less than 15%, less than 20%, or less than 25% when the drug is administered.

“Subject” as used herein can mean a mammal that wants or is in need of the herein described assays or methods. The subject may be a patient. The subject may be a human or a non-human animal. The subject may be a mammal. The mammal may be a primate or a non-primate. The mammal can be a primate such as a human. The mammal can be a non-primate such as, for example, dog, cat, cow, pig, mouse, rat, camel, llama, hedgehog, anteater, platypus, elephant, alpaca, horse, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant. The subject may be male or female. In some embodiments, the subject has a specific genetic marker.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

The terms “transformation” and “transfection” as used herein refer to the introduction of foreign DNA or polynucleotide into prokaryotic or eukaryotic cells. Transformation of prokaryotic cells may be accomplished by a variety of means known to the art including, for example, the treatment of host cells with CaCl₂ to make competent cells, electroporation, etc. Transfection of eukaryotic cells may be accomplished by a variety of means known to the art including, for example, calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

“Variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequence substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

### 2. DNA FRAGMENT

One or more DNA fragments are identified from a first cell type. Each DNA fragment is, for example, about 25 nt to about 200 nt in length, about 50 nt to about 150 nt in length, about 75 nt to about 125 nt in length, about 100 nt to about 200 nt in length, or about 50 nt to about 100 nt in length. Each DNA fragment is present in a genomic region comprising an epigenetic feature in the first cell type. The epigenetic feature may be, for example, open chromatin, a histone mark, or DNA methylation. Histone marks may include, for example, methylation or acetylation of an amino acid residue of a histone subunit. Each DNA fragment is selected as being specific for the first cell type if the same epigenetic feature is not present in the same genomic region in one or more second cell types. The DNA fragments may be identified by comparing DNAse hypersensitivity data for the first cell type to DNAse hypersensitivity data for the one or more second cell types. DNAse hypersensitivity data can identify the regions of open chromatin in a cell or tissue. Open chromatin may indicate a location in the genome of protein-binding and regulatory activity. Regions that may be sensitive to DNAse may include, for example, gene promoters, enhancers, silencers, insulators, locus control regions, and meiotic recombination hot spots. The DNAse hypersensitivity data may be obtained by methods such as DNAse-seq or ATAC-seq. Alternatively, the DNA fragments may be identified by comparing histone modification data, such as acetylation of the lysine amino acid that is the 27^{th} residue of histone subunit 3 (H3K27ac), data for the first cell type to histone modification data for one or more second cell types. The histone modification data may be obtained by methods such as ChIP-seq. The DNA fragments may be identified by comparing DNA methylation. Bisulfite sequencing may be used to identify regions of DNA methylation. The DNA fragments may be identified by comparing Self-transcribing active regulatory region sequencing (STARR-seq) data. STARR-seq is a method that may be used to identify sequences in the genome that act as transcriptional enhancers.

The first cell type and second cell type may each independently be a cardiac cell, skeletal muscle cell, smooth muscle cell, endothelial cell, intestinal cell, epithelial cell, liver cell such as parenchymal cell (also known as hepatocyte), adipocyte, fibroblastic cell, Kupffer cell, stromal cell, retinal cell, hematopoietic stem cell, satellite cell, CNS cell, astrocyte, glial cell, brain cell, neuronal cell, or neuronal subtype cell such as dopaminergic neuron, gabaergic neuron, and glutamatergic neuron. In some embodiments, the first or second cell type is a cardiomyocyte. In some embodiments, the first or second cell type is a skeletal myoblast. In some embodiments, the epigenetic feature is not present in the same genomic region in at least two second cell types. In some embodiments, the epigenetic feature is not present in the same genomic region in at least one second cell type but is present in the same genomic region in at least one third cell type. In some embodiments, the first cell type and the second cell type are from different tissues. In some embodiments, the first cell type and the second cell type are different cell types. In some embodiments, the one or more DNA fragments are sequenced. DNA sequencing may be completed by any suitable method known by one of skill in the art. In some embodiments, the one or more DNA fragments are synthesized. DNA synthesis may be completed by any suitable method known by one of skill in the art.

### 3. PROMOTER SEQUENCE

A promoter sequence may comprise at least one DNA fragment. In some embodiments, the promoter sequence comprises a combination of at least about 2, at least about 3, or at least about 4 of the one or more DNA fragments. The DNA fragments may be arranged in any order in the promoter sequence. The DNA fragments may be present in the same reading frame in the promoter sequence. The DNA fragments may be directly adjacent to one another in the promoter sequence. The DNA fragments may be adjacent to one another with a spacer of 1 to 3, or 1 to 5, or 1 to 10, nucleotides independently between the DNA fragments in the promoter sequence. DNA fragments may be operably linked in the promoter sequence.

The compositions and methods detailed herein may be used to identify promoter sequences directing tissue-specific or cell-specific gene expression, such as expression specific for a single tissue or cell type but not others, and/or expression specific for multiple tissue or cell types but not others. The promoter may direct specific gene expression in any cell type, tissue, disease state, or as a result of any pharmacological stimulus. The tissue-specific or cell-specific promoter may also be referred to as a tissue-specific or cell-specific regulatory element. The tissue-specific or cell-specific promoter comprises a polynucleotide sequence for directing tissue-specific or cell-specific gene expression. In some embodiments, the tissue-specific or cell-specific promoter is a cardiac muscle-specific promoter. In some embodiments, the tissue-specific or cell-specific promoter is a skeletal muscle-specific promoter. In some embodiments, the tissue-specific or cell-specific promoter directs gene expression in cardiomyocytes but not in skeletal myoblasts. In some embodiments, the tissue-specific or cell-specific promoter directs gene expression in skeletal myoblasts but not in cardiomyocytes.

### 4. VECTORS

One or more promoter sequence may be inserted or incorporated into a vector. The vector may comprise at least one promoter sequence. The vector may comprise at least one tissue-specific or cell-specific promoter. As used herein, the term “vector” refers to a polynucleotide that transfers polynucleotide segment(s) from one cell to another. A vector may also be referred to as a “vehicle” and is a type of “polynucleotide construct” or “nucleic acid construct.” A vector may be a viral vector, bacteriophage, bacterial artificial chromosome, or yeast artificial chromosome. A vector may be a DNA or RNA vector. A vector may be a self-replicating extrachromosomal vector. Vectors include circular nucleic acid constructs such as plasmids, cosmids, etc., as well as linear nucleic acid constructs (e.g., lambda, phage constructs, PCR products), viruses, lentiviruses, AAV, and other mediums. A vector may include an origin of replication. A vector may include expression signals such as a promoter and/or an enhancer, and in such a case it is referred to as an expression vector. The term “expression vector” as used herein refers to a polynucleotide molecule containing a desired coding sequence and appropriate polynucleotide sequences necessary for the expression of the operably linked coding sequence in a particular host organism. The expression vector may contain one or more polynucleotide sequences that generally have some function in the replication, maintenance, or integrity of the vector, such as, for example, origins of replication, as well as one or more selectable marker genes. The expression vector can be transfected and into an organism to express a gene. The expression vector may be recombinant. A polynucleotide sequence for encoding a desired protein can be inserted or introduced into an expression vector. A vector may include polynucleotide sequences to promote or control expression in prokaryotes such as a promoter, an operator (optional), and a ribosome binding site, and other sequences. A vector may include polynucleotide sequences to promote or control expression in eukaryotes such as a promoter, enhancers, termination signal, and polyadenylation signal. The polynucleotide sequence of the vector may be operably linked to another polynucleotide sequence in the vector using conventional recombinant DNA techniques. The vector may be any suitable vector known in the art such as, for example, a lentiviral or adeno-associated viral (AAV) vector. For example, the vector may be a AAV8 or AAV9 vector. In some embodiments, the AAV vector is a modified AAV vector. The modified AAV vector may have enhanced cardiac and/or skeletal muscle tissue tropism. The modified AAV vector may be capable of delivering and expressing a gene in the cell of a mammal. For example, the modified AAV vector may be an AAV-SASTG vector (Piacentino et al. Human Gene Therapy 2012, 23, 635-648). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

The vector may comprise one promoter sequence. The vector may comprise at least one promoter sequence. The vector may comprise multiple promoter sequences. The vector may include at least 1 promoter sequence. The vector may include a combination of at least about 2, at least about 3, or at least about 4 promoter sequences. The vector may include at least 1 DNA fragment. The vector may include a combination of at least about 2, at least about 3, or at least about 4 DNA fragments.

The vector may further include at least one sequence tag. A sequence tag comprises a polynucleotide sequence of at least about 5 nt or bp, at least about 6 nt or bp, at least about 7 nt or bp, at least about 8 nt or bp, at least about 9 nt or bp, at least about 10 nt or bp, at least about 11 nt or bp, at least about 12 nt or bp, at least about 13 nt or bp, at least about 14 nt or bp, at least about 15 nt or bp, less than about 20 nt or bp, less than about 19 nt or bp, less than about 18 nt or bp, less than about 17 nt or bp, less than about 16 nt or bp, less than about 15 nt or bp, less than about 14 nt or bp, less than about 13 nt or bp, less than about 12 nt or bp, less than about 11 nt or bp, or less than about 10 nt or bp in length, that is specific for the DNA fragment. A sequence tag may be specific for one DNA fragment. For example, a promoter sequence made up of multiple DNA fragments may be associated with multiple sequence tags. In some embodiments, the sequence tag is about 10 nt or bp in length. The sequence tag may be randomly assigned to each DNA fragment. The sequence tag may be referred to as a “barcode.”

The vector may further include at least one polynucleotide encoding a reporter. The reporter is capable of generating a detectable signal. In some embodiments, the signal from the reporter is a fluorescent signal. Reporters may include, for example, fluorescent proteins. Fluorescent proteins may include, for example, luciferase and green fluorescent protein (GFP). GFP and its numerous related fluorescent proteins are in widespread use as protein tagging agents (for review, see Verkhusha et al., 2003, GFP-like fluorescent proteins and chromoproteins of the class Anthozoa. In: Protein Structures: Kaleidescope of Structural Properties and Functions, Ch. 18, pp. 405-439, Research Signpost, Kerala, India). GFP-like proteins are an expanding family of homologous, 25-30 kDa polypeptides sharing a conserved beta-strand “barrel” structure. The GFP-like protein family currently comprises well over 100 members, cloned from various Anthozoa and Hydrozoa species, and may include red, yellow, and green fluorescent proteins and a variety of non-fluorescent chromoproteins. A wide variety of fluorescent protein labeling assays and kits are commercially available, encompassing a broad spectrum of GFP spectral variants and GFP-like fluorescent proteins, such as DsRed and other red fluorescent proteins (Clontech, Palo Alto, Calif.; Amersham, Piscataway, N.J.). In some embodiments, the reporter is GFP. The promoter may be operably linked to the polynucleotide encoding a reporter. The promoter may be upstream of and operably linked to the polynucleotide encoding a reporter. The promoter and the polynucleotide encoding a reporter may both be upstream of the sequence tag. The vector may include at least one DNA fragment, and, for example, up to 3 or 4 or 5 DNA fragments in succession adjacent to one another with or without spacers, together forming a promoter, upstream of and operably linked to the polynucleotide encoding a reporter, such as one GFP. The level of expression of the reporter may be determined by a variety of methods known in the art, such as, for example, flow cytometry.

One or more vectors may be transduced into an expression cell. In some embodiments, the expression cell is of the same cell type as the first cell type. The vector may be transduced or transfected into cells by any suitable method known in the art. In some embodiments, a cell is transfected with a vector by electroporation. The vector may be expressed or transcribed in the expression cell.

The level of transcription of the sequence tag in the expression cell may be determined. Examples of methods or processes used to determine transcription levels may include, for example, nucleic acid hybridization, Northern blotting, in situ hybridization, RNAse protection assays, microarrays, RNA sequencing (RNAseq), quantitative polymerase chain reaction (or other nucleic acid replication reactions), a NanoString nCounter platform, reverse transcription polymerase chain reaction (RT-PCR), sequencing such as nucleic acid sequencing, ligase chain reaction (LCR), multiplex ligation-dependent probe amplification, transcription-mediated amplification (TMA), strand displacement amplification (SDA), nucleic acid sequence based amplification (NASBA), protein product detection, and visible light or ultra-violet light spectrophotometry or diffraction, or a combination thereof. Such methods can utilize fluorescent dyes, chemiluminescent dyes, radioactive tracers, enzymatic reporters, dye molecules, chemical reaction products, or other means of reporting the amounts or concentrations of nucleic acid molecules or peptides. Oligonucleotide probes may be used to detect the presence of complementary target sequences by hybridization with target sequences. In some embodiments, the level of transcription of the sequence tag in the expression cell is determined by quantitative DNA sequencing. In some embodiments, the level of transcription of the sequence tag in the expression cell is determined by Illumina sequencing.

An increased level of transcription of the sequence tag in the expression cell relative to a control may indicate that the promoter sequence directs tissue-specific or cell-specific gene expression in the tissue and/or cell corresponding to the expression cell type. The control may include, for example, a transcription level of the same sequence tag in an expression cell from a different tissue or cell type, a transcription level of a different sequence tag in the same expression cell type, or a transcription level of a different sequence tag in an expression cell from a different tissue or cell type. In some embodiments, the level of transcription of the sequence tag in the expression cell is compared to the level of transcription of a different sequence tag corresponding to a different promoter sequence. In some embodiments, the level of transcription of the sequence tag is compared to the level of transcription of the same sequence tag in a different cell type.

Further provided is a vector comprising at least one tissue-specific or cell-specific promoter sequence identified by a method as detailed herein. A plurality of vectors may be combined or gathered to form a regulatory element fragment library. In some embodiments, the regulatory element fragment library comprises vectors with putative promoter sequences. In some embodiments, the regulatory element fragment library comprises vectors with confirmed tissue-specific and/or cell-specific promoter sequences. In some embodiments, the regulatory element fragment library comprises vectors with a combination of putative and confirmed tissue-specific and/or cell-specific promoter sequences.

### 5. ADMINISTRATION

The vectors as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the vector may be delivered by mRNA delivery and ribonucleoprotein (RNP) complex delivery. The vector may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000. In some embodiments, the transfection efficiency may be kept low in an effort to result in approximately one vector per cell.

The vectors may be administered to a subject. Such compositions can be administered in dosages and by techniques well known to those skilled in the medical arts taking into consideration such factors as the age, sex, weight, and condition of the particular subject, and the route of administration. The vector may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the vector is administered to a subject intramuscularly, intravenously, or a combination thereof. The vector may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The vector may be injected into the brain or other component of the central nervous system. The composition may be injected into the skeletal muscle or cardiac muscle. For example, the composition may be injected into the tibialis anterior muscle or tail. For veterinary use, the vector may be administered as a suitably acceptable formulation in accordance with normal veterinary practice. The veterinarian may readily determine the dosing regimen and route of administration that is most appropriate for a particular animal. The vector may be administered by traditional syringes, needleless injection devices, “microprojectile bombardment gone guns,” or other physical methods such as electroporation (“EP”), “hydrodynamic method”, or ultrasound.

Upon delivery of the presently disclosed vector into the cells of the subject, the transfected cells may express the gene, such as a gene encoding a reporter protein, that is operably linked to the DNA fragment or promoter as detailed herein.

a. Cell Types

Any of the delivery methods and/or routes of administration detailed herein can be utilized with a myriad of cell types, for example, those cell types currently under investigation for cell-based therapies, including, but not limited to, immortalized myoblast cells, such as wild-type and DMD patient derived lines, primal DMD dermal fibroblasts, stem cells such as induced pluripotent stem cells, bone marrow-derived progenitors, skeletal muscle progenitors, human skeletal myoblasts from DMD patients, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells. Immortalization of human myogenic cells can be used for clonal derivation of genetically corrected myogenic cells. Cells can be modified ex vivo to isolate and expand clonal populations of immortalized DMD myoblasts that include a genetically corrected or restored dystrophin gene and are free of other nuclease-introduced mutations in protein coding regions of the genome.

### 6. METHODS OF IDENTIFYING A PROMOTER SEQUENCE FOR DIRECTING TISSUE-SPECIFIC OR CELL-SPECIFIC GENE EXPRESSION

Provided herein are methods of identifying a promoter sequence for directing tissue-specific or cell-specific gene expression. The method may include identifying one or more DNA fragments from a first cell type, wherein each DNA fragment is, for example, 50 nt to 200 nt in length and present in a genomic region comprising at least one epigenetic feature in the first cell type. The epigenetic feature may be selected from open chromatin and histone mark and DNA methylation. The epigenetic feature may not present be in the same genomic region in at least one second cell type. The method may further include inserting into a vector at least one promoter sequence comprising at least one of the one or more DNA fragments and at least one sequence tag, wherein the sequence tag comprises a polynucleotide sequence that is specific for each DNA fragment. In some embodiments, the vector further includes a polynucleotide encoding a reporter downstream of and operably linked to the at least one promoter sequence. The method may further include Transducing one or more vectors into an expression cell. The method may further include determining the level of transcription of the sequence tag in the expression cell. In some embodiments, an increased level of transcription of the sequence tag in the expression cell relative to a control indicates that the promoter sequence directs tissue-specific or cell-specific gene expression. In some embodiments, an increased or decreased level of expression of the reporter in the expression cell relative to a control indicates that the promoter sequence directs tissue-specific or cell-specific gene expression. A control may be, for example, an expression cell with a vector without the promoter sequence. In some embodiments, the method further includes sequencing the one or more DNA fragments prior to inserting into a vector the at least one promoter sequence. In some embodiments, the method further includes synthesizing the one or more DNA fragments prior to inserting into a vector the at least one promoter sequence. In some embodiments, the first cell and the second cell are from different tissues. In some embodiments, the first cell and the second cell are different cell types. In some embodiments, the method further comprises comparing the level of transcription of the sequence tag in the expression cell to the level of transcription of a different sequence tag corresponding to a different promoter sequence. In some embodiments, the method further comprises comparing the level of transcription of the sequence tag in the expression cell to the level of transcription of the same sequence tag in a different or another cell type.

### 7. EXAMPLES

### Example 1

### Large Scale Screening and Analysis of Gene Regulatory Systems

CRISPR screens were used for the cloning and downstream analysis of exceptionally large (>10⁸) plasmid libraries. A method for mapping functional regulatory elements using CRISPR/Cas9 epigenome editing was developed (Klann, T. S. et al. Nat. Biotechnol. 2017, 35, 561-568) and includes some general techniques similar to those used to generate the libraries as detailed herein. In order to perform these screens, libraries were designed and cloned into lentiviral vectors in a pooled fashion. The ability to clone large scale libraries and generate sufficient quality lentivirus was used in the disclosed screening approach.

### Example 2

### Adeno-Associated Viral Delivery In Vivo

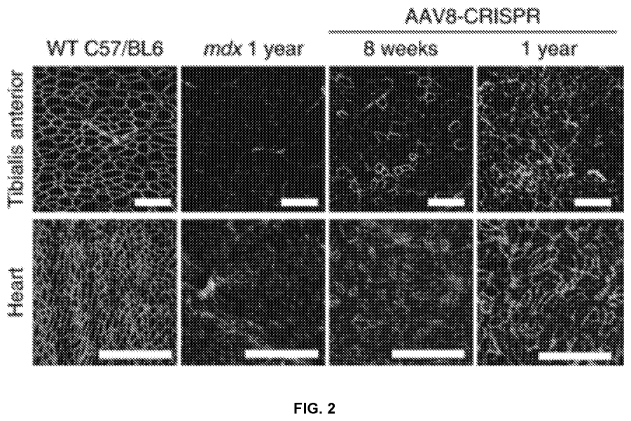

AAV delivery in mice has been used for the systemic treatment of Duchenne Muscular dystrophy (DMD). Utilizing a dual vector approach, CRISPR-Cas9 mediated genome engineering was used to remove a premature stop codon and restore a truncated version of the dystrophin gene (FIG. 2)(Nelson, C. E. et al. Nat. Med. 2019, doi:10.1038/s41591-019-0344-3; Nelson, C. E. et al. Science 2016, 351, 403-407). AAV8 and AAV9 were potent transducers of cardiac and skeletal muscle organism-wide. In addition, CRISPR-based transcriptional repressors were delivered to adult mice in order to repress PCSK9 in the liver, resulting in a reduction in circulating LDL cholesterol levels (Thakore, P. I. et al. Nat. Commun. 2018, 9, 1674). AAV vectors were used to successfully deliver genes to cells, both in culture and in vivo, and direct expression of the genes, similar to the vector libraries as detailed herein.

### Example 3

### Design of AAV and Lentiviral Plasmids for Combinatorial Libraries of Regulatory Elements

Plasmids were designed and built for the iterative addition of custom designed and synthesized DNA fragments in order to build novel promoter elements. Plasmids for AAV and lentivirus were cloned and sequenced. In order to test that the cloning strategy was feasible, fragments of the widely used EFS constitutive promoter were generated, and the promoter was rebuilt through the same iterative process as used for the promoter libraries detailed herein. A plasmid containing the complete EFS promoter was constructed, as well as a GFP transgene under its control, to demonstrate that significant levels of expression could be achieved. This demonstrated that the small DNA “scars” generated by the molecular cloning process did not adversely affect gene expression.

### Example 4

### Generation of Regulatory Modules to Drive Gene Expression in any Cell Type

The methodology for identification of novel combinations of regulatory sequences that control gene expression from AAV vectors in specific tissues in vivo, was used to generate a range of regulatory “modules” capable of driving transgene expression in any cell type. A combinatorial library of >10⁰ regulatory elements was generated and derived from data on tissue-specific region of open chromatin—a signature of gene regulatory activity—and library members with particular in vivo activity will be selected.

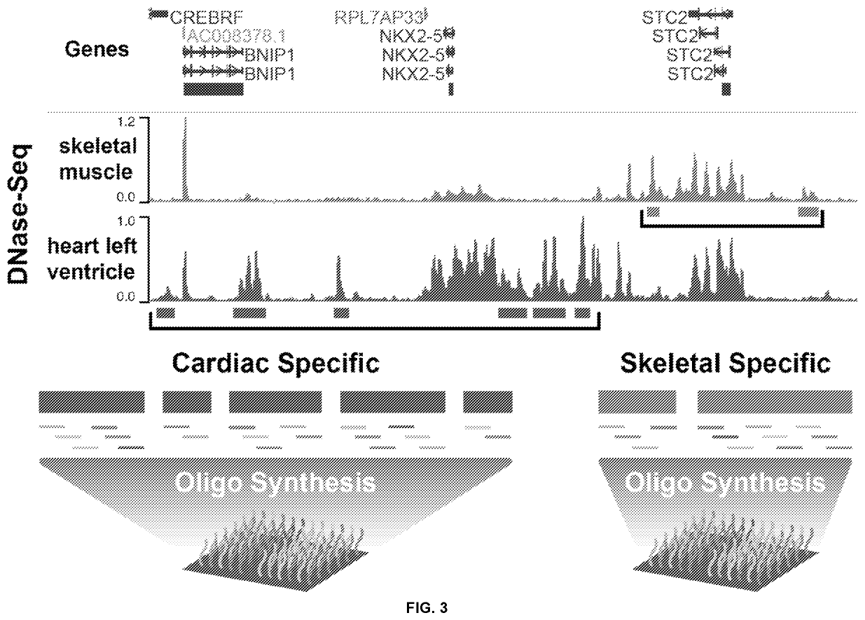

Design and synthesis of regulatory sequence libraries. Libraries were designed by generating novel combinations of DNA fragments derived from tissue specific DNase-hypersensitive sites (DHS). Tissue specificity was determined by the comparison of skeletal muscle DHS peaks to cardiac muscle DHS peaks and eliminating peaks that are shared between other tissue types (FIG. 3). These peaks were divided into small, 100-150 bp fragments that have overlap between each fragment. Each fragment was synthesized with a barcode that was identified via deep sequencing prior to combinatorial assembly. High-throughput sequencing has been performed to verify the complexity of the skeletal muscle (99,791 fragments of 145 bp) and cardiac muscle (149,934 fragments of 145 bp) libraries.

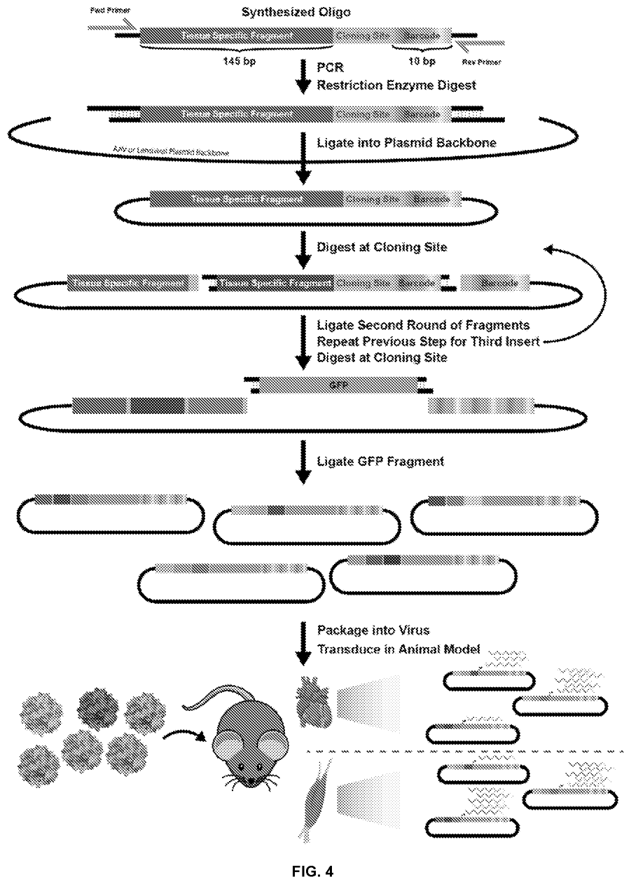

Cloning of regulatory sequence combination libraries. Lentiviral and AAV libraries were generated for cell culture or in vivo mouse experiments. Libraries were cloned using a nested modular cloning strategy to allow for increasingly complex libraries to be generated (FIG. 4). In addition to increased regulatory module complexity, the design of these vectors was modular for the exchange of any transgene.

identification of potent novel regulatory “modules”. Lentiviral libraries were transduced into human cardiomyocyte or skeletal muscle cell lines. Transduced cells were either collected en masse or sorted by flow cytometry for GFP expression, and mRNA was collected. Using high-throughput sequencing and transcript-specific RT-PCR primers, the most potent drivers of expression were identified based on barcode representation. By comparing the results derived from each cell type, specific regulatory sequences that are highly represented in cardiac cells, but not skeletal myoblasts, and vice versa, were selected. In conjunction with these in vitro experiments, AAV libraries were injected systemically by intravenous administration to adult mice. After 2 weeks, cardiac and skeletal muscle, as well as liver, tissues were collected, RNA isolated, and high-throughput sequencing performed. Based on the representation of barcodes found in the sequencing, the regulatory sequences that were responsible for driving gene expression in each tissue were inferred. In addition to muscle selectivity, a lack of expression in off-target tissues such as the liver was be examined in these analyses. The regulatory sequences driving high expression in skeletal or cardiac muscle in combination with no or low signal in liver represent the population of sequences used for further validation.

Validation of novel regulatory “modules”. Novel regulatory modules are tested in vivo for their ability to drive expression in a tissue-specific manner. Regulatory modules are cloned individually into AAV driving expression of either GFP or a luciferase transgene. Plasmids are packaged into AAV9 for efficient delivery to both cardiac and skeletal muscle. Expression levels are compared between combinatorial regulatory sequences using qRT-PCR to determine the levels of expression. If a large number of sequences that drive specific expression is identified in the initial screen, a smaller pooled screening approach is used to validate these hits in batches. A series of tissue-specific regulatory element combinations that drive a range of gene expression levels are examined.

Future work will build on the framework detailed herein to expand cell type specificity and explore the potential for generating inducible regulatory “modules” that respond to small molecules, enabling pharmacologically controlled gene therapy.

### Example 5

### Generation of Skeletal and Cardiac Muscle Libraries

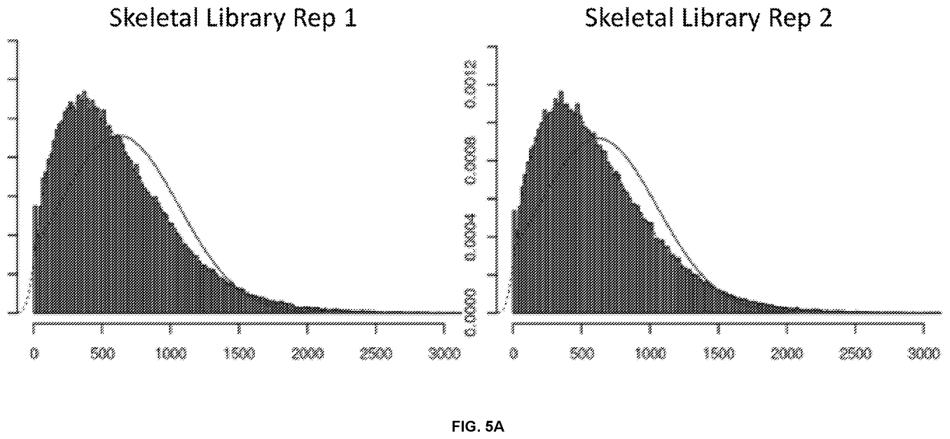

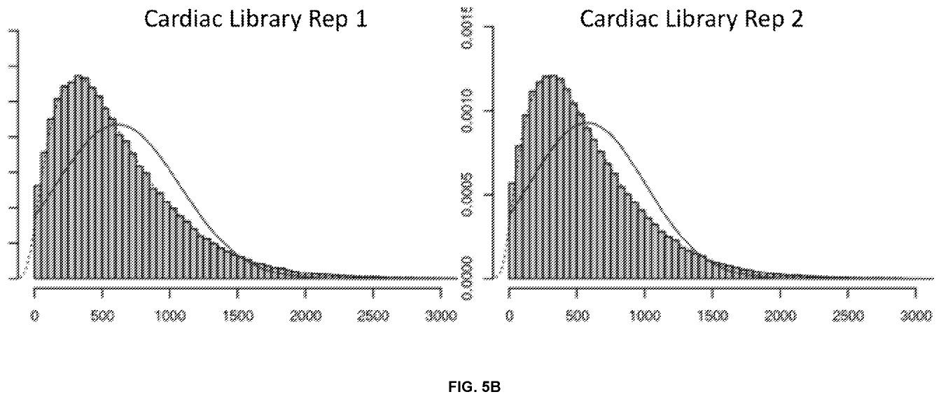

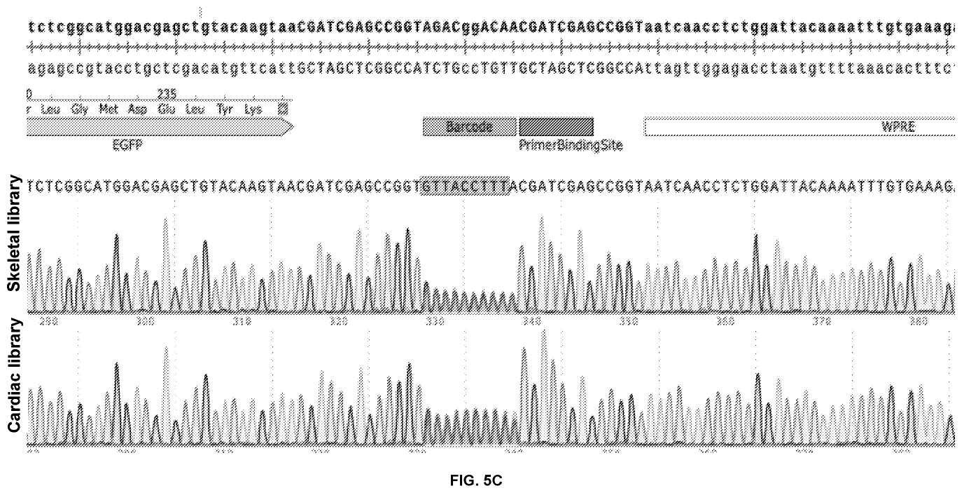

A library of potential tissue-specific promoters from skeletal muscle was generated. The library contained 99,791 unique DNA fragments, each DNA fragment being 145 bp in length. Each DNA fragment was obtained from open chromatin regions specific to skeletal muscle. The DNA fragments were tiled across approximately 36,000 unique chromosomal regions of open chromatin. The average size of the open chromatin region was 250 bp. Once the library of 145 bp DNA fragments were identified, oligonucleotides corresponding to each 145 bp DNA fragment were synthesized. The library of 145 bp DNA fragments was then inserted into AAV vectors (FIG. 4). Each DNA fragment was inserted upstream of and operably linked to a polynucleotide encoding GFP. Each vector included at least one DNA fragment, forming a promoter, upstream of and operably linked to the polynucleotide encoding one GFP. A barcode or sequence tag, each specific to a single DNA fragment, was included in the vector downstream of the polynucleotide encoding GFP. The collection of vectors, referred to as the skeletal muscle library, was sequenced to confirm the presence of the 99,791 unique DNA fragments in the library. FIG. 5A shows the distribution of the vector sequences (y-axis is the frequency; x-axis is the number of fragments with the corresponding frequency of sequencing reads), which confirms at least 99.9% of the 99,791 unique DNA fragments are indeed included in the library.

A library of potential tissue-specific promoters from cardiac muscle was generated. The library contained 149,934 unique DNA fragments, each DNA fragment being 145 bp in length. Each DNA fragment was obtained from open chromatin regions specific to cardiac muscle. The DNA fragments were tiled across approximately 28,000 unique chromosomal regions of open chromatin. The average size of the open chromatin region was 216 bp. Once the library of 145 bp DNA fragments were identified, oligonucleotides corresponding to each 145 bp DNA fragment were synthesized. The library of 145 bp DNA fragments was then inserted into AAV vectors (FIG. 4). Each DNA fragment was inserted upstream of and operably linked to a polynucleotide encoding GFP. Each vector included at least one DNA fragment, forming a promoter, upstream of and operably linked to the polynucleotide encoding one GFP. A barcode or sequence tag, each specific to a single DNA fragment, was included in the vector downstream of the polynucleotide encoding GFP. The collection of vectors, referred to as the cardiac muscle library, was sequenced to confirm the presence of the 149,934 unique DNA fragments in the library. FIG. 5B shows the distribution of the vector sequences (y-axis is the frequency; x-axis is the number of fragments with the corresponding frequency of sequencing reads), which confirms at least 99.9% of the 149,934 unique DNA fragments are indeed included in the library.

The vectors in the skeletal library and the in the cardiac library were also sequenced at the region of the barcode in the vector. As indicated above and as shown in FIG. 5C, a barcode or sequence tag, each specific to a single DNA fragment, was included in the vector downstream of the polynucleotide encoding GFP. Among the population of vectors, the sequence of the polynucleotide encoding GFP is the same, as well as the sequence immediately downstream of the barcode. As shown in FIG. 5C, the average sequencing results of the barcode from the mixture of vectors in each library reveals an even distribution of nucleotides, indicating that each DNA fragment in the vectors can be distinguished.

### Example 6

### Plasmid Transfection of Human Cells

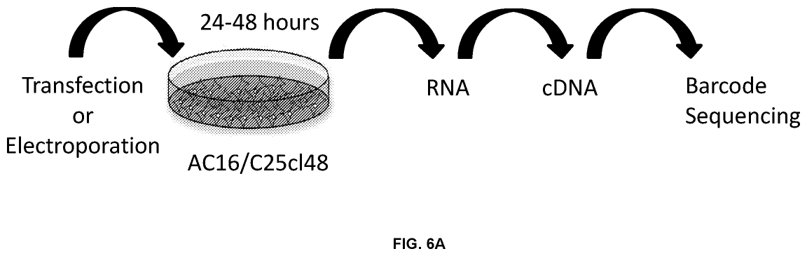

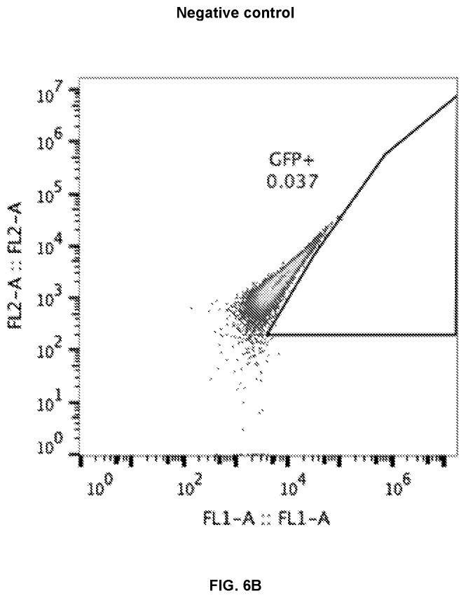

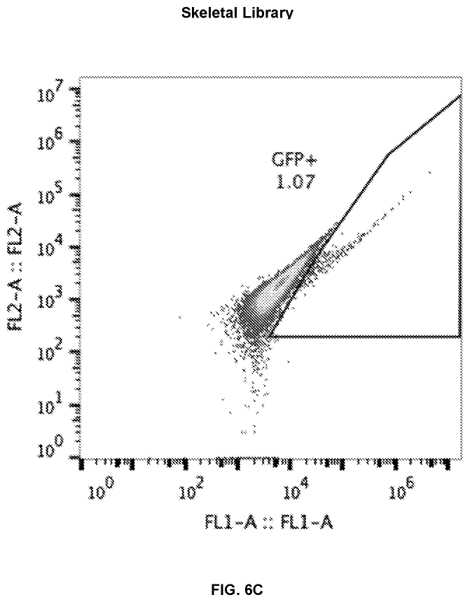

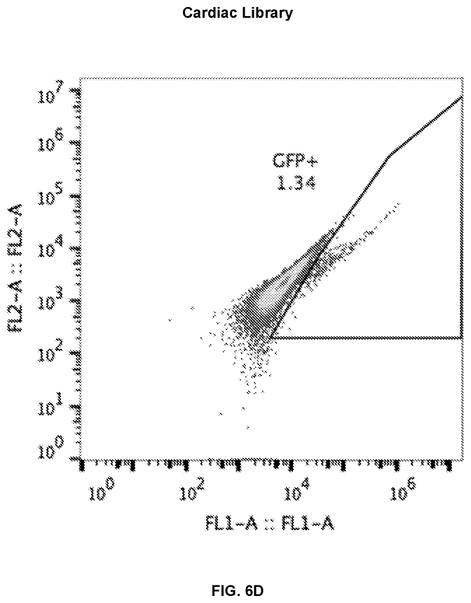

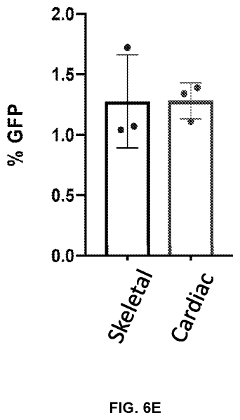

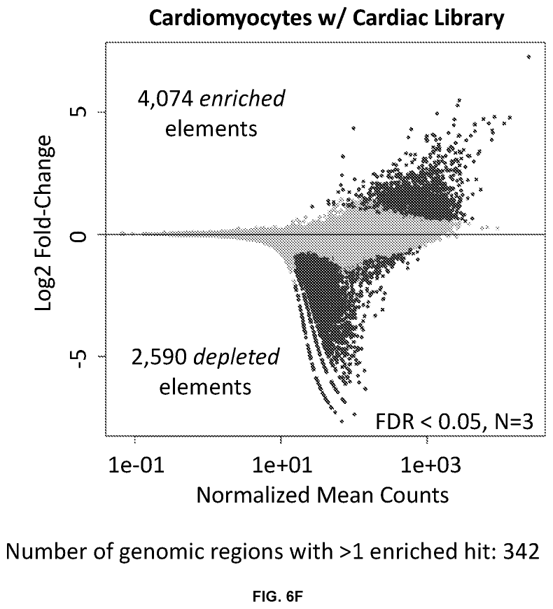

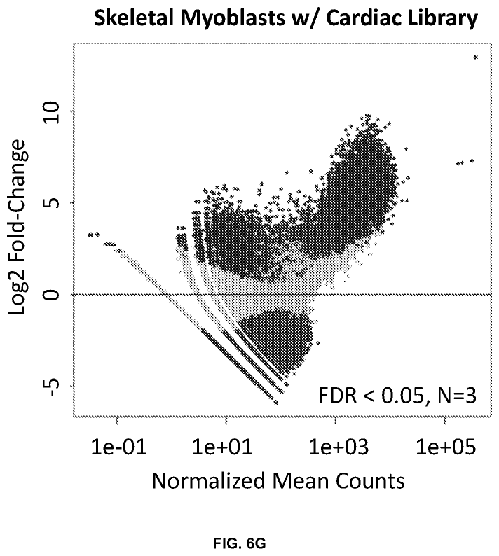

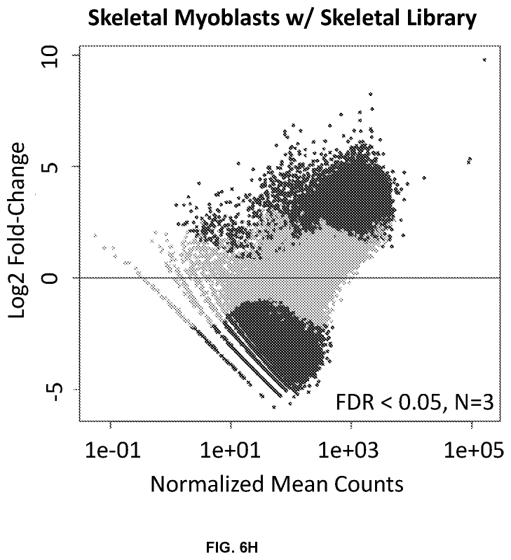

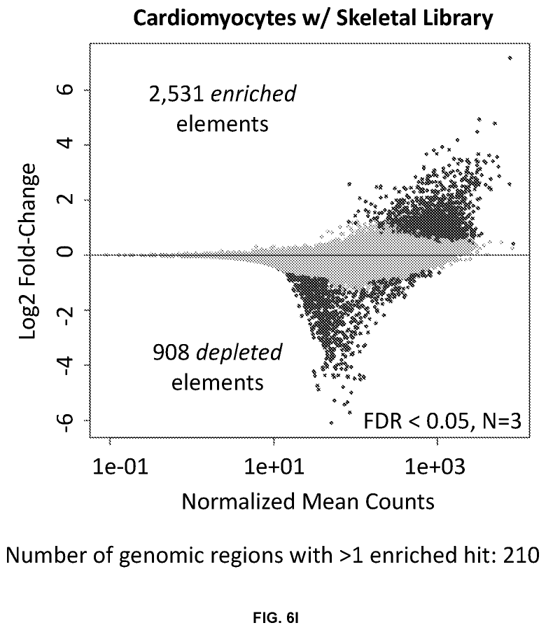

Human cells (C25cl48 cells) were transfected with the skeletal muscle library described in Example 5 (FIG. 6A). C25cl48 cells are skeletal myoblasts from an immortalized human myoblast cell line. Some cells may have included more than one vector, but as detailed in Example 5, expression from each vector was able to be distinguished using the barcode or sequence tag specific for each DNA fragment in the vector.

Human cells (AC16 cells) were transfected with the cardiac muscle library described in Example 5 (FIG. 6A). AC16 is a human cardiomyocyte cell line, and the cell are pre-contractile and exhibit similar cardiac gene expression profiles as primary human cardiomyocytes. Some cells may have included more than one vector, but as detailed in Example 5, expression from each vector was able to be distinguished using the barcode or sequence tag specific for each DNA fragment in the vector.

The AC16 cardiomyocyte cells with either a negative control library, the skeletal library, or the cardiac library, were examined for GFP expression with flow cytometry (FIGS. 6B-6E). The negative control was a vector encoding GFP without any DNA fragments included. Approximately 1% of the cells received promoters that drove GFP expression in cardiomyocytes, indicating that the system worked to use GFP expression to report promoter function.

GFP expression was then analyzed in cardiomyocytes transduced with the cardiac muscle library (FIG. 6F), in skeletal myoblasts transduced with the cardiac muscle library (FIG. 6G), in skeletal myoblasts transduced with the skeletal muscle library (FIG. 6H), and in cardiomyocytes transduced with the skeletal muscle library (FIG. 6I). The barcodes were sequenced and counted, and all the vectors that were transduced into the cells were compared to the vectors that resulted in expression of GFP in the cell. For FIGS. 6F-6I, each dot represents one barcode/DNA fragment, and the gray dots represent the barcodes/DNA fragments that did not have an effect on GFP expression. The black dots represent the barcodes/DNA fragments that did affect GFP expression, with the ones above the zero line increasing GFP expression, and the ones below the zero line decreasing GFP expression.

As shown in FIG. 6F, 4,074 DNA fragments from the cardiac library resulted in increased GFP expression in cardiac cells. 2,590 DNA fragments from the cardiac library resulted in decreased GFP expression in cardiac cells.

As shown in FIG. 6I, 2,531 DNA fragments from the skeletal library resulted in increased GFP expression in cardiac cells. 908 DNA fragments from the skeletal library resulted in decreased GFP expression in cardiac cells.

Comparison of the cardiomyocytes (FIG. 6F) and the skeletal myoblasts with the cardiac library (FIG. 6G) indicates that of the 4,074 DNA fragments from the cardiac library that resulted in increased GFP expression in cardiac cells, 845 of them turned off GFP expression in skeletal cells. 845 cardiac elements were enriched in cardiomyocytes and depleted in skeletal myoblasts. That is, each of these 845 DNA fragments from the cardiac library may form a promoter that drives gene expression specific for cardiomyocytes and not skeletal myoblasts.

Comparison of the skeletal myoblasts (FIG. 6H) and the cardiomyocytes with the skeletal library (FIG. 6I) indicates that of the DNA fragments from the skeletal library that resulted in increased GFP expression in skeletal myoblasts, 236 of them turned off GFP expression in cardiomyocytes. 236 skeletal elements were enriched in skeletal myoblasts and depleted in cardiomyocytes. That is, each of these 236 DNA fragments from the skeletal library may form a promoter that drives gene expression specific for skeletal myoblasts and not cardiomyocytes.

### Example 7

### AAV Viral Transduction in Mice

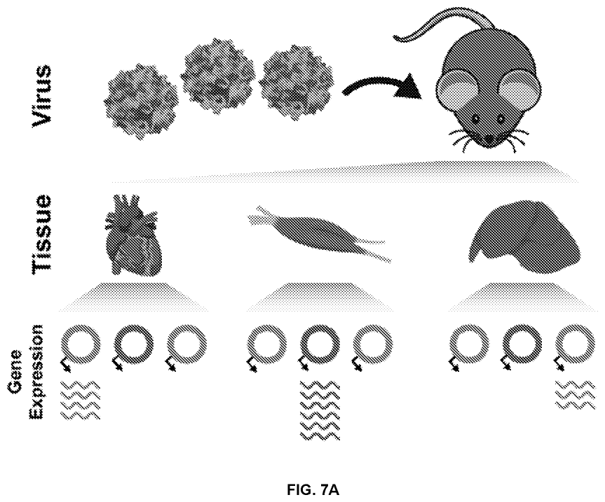

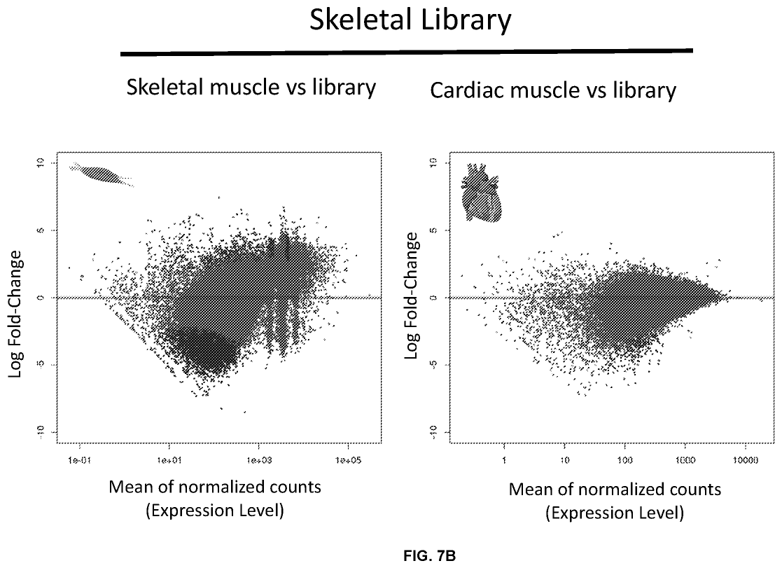

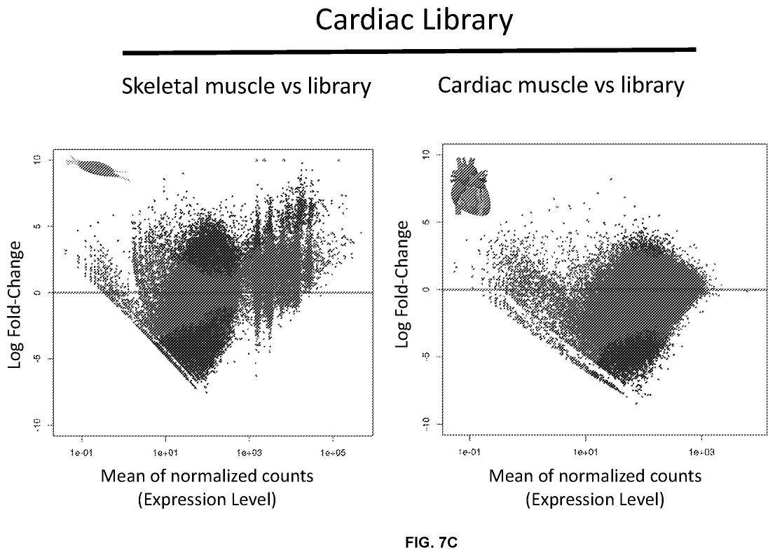

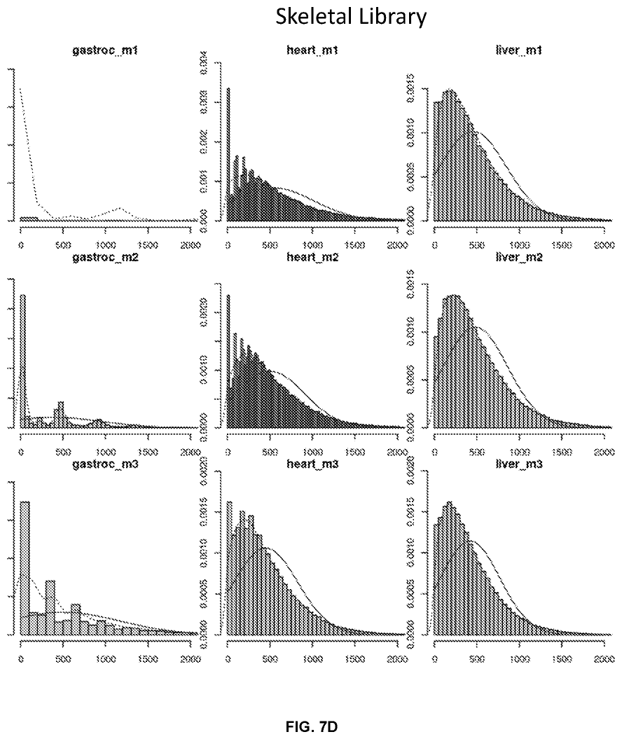

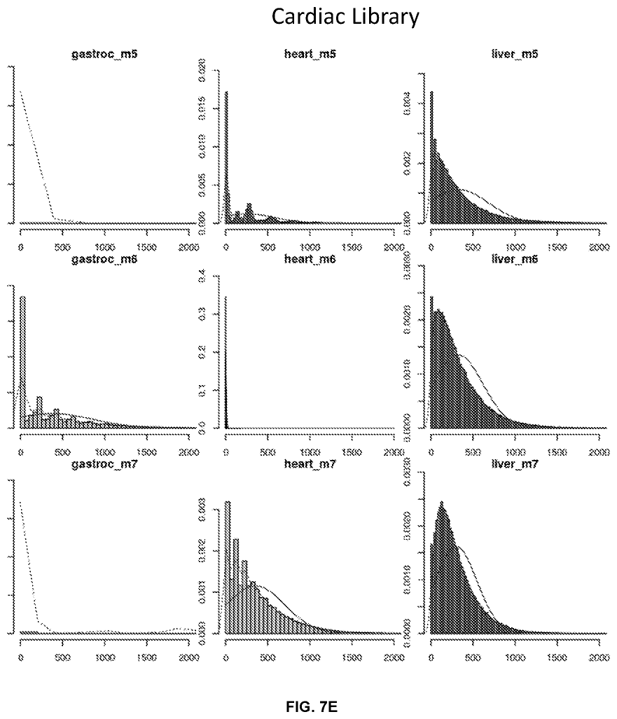

Mice were transfected with the cardiac muscle library or the skeletal muscle library described in Example 5 (FIG. 7A). Each mouse was injected systemically with 6.6e11-1.5e12 vg of AAV vector, and tissue was harvested after 7 days. Expression of GFP was examined for each. The barcodes were sequenced and counted, and all the vectors that were transduced into the cells were compared to the vectors that resulted in expression of GFP in the cell. In the graphs for the skeletal library (FIG. 7B) and the cardiac library (FIG. 7C), each dot represents one barcode/DNA fragment, and the gray dots represent the barcodes/DNA fragments that did not have an effect on GFP expression. The black dots represent the barcodes/DNA fragments that did affect GFP expression, with the ones above the zero line increasing GFP expression, and the ones below the zero line decreasing GFP expression, similar to the results for cell culture described in Example 6.

Of the barcodes/DNA fragments that did affect GFP expression in mice from FIG. 7B and FIG. 7C, the distribution of the expression among various tissues was examined by deep sequencing of the expressed barcodes that correspond to DNA fragments in the promoter of the vector. As shown in FIG. 7D, various DNA fragments from the skeletal library positively and negatively affected expression of GFP across different tissues, as did DNA fragments from the cardiac library (FIG. 7E).

The sequences of the DNA fragments and whether or not they affected GFP expression were examined, assisted by algorithms for identifying known sequence motifs of DNA-binding proteins such as human transcription factors. The analysis indicated that some DNA regions that were open in skeletal muscle and closed in cardiac muscle, but still increased expression of GFP in cardiac cells, included elements or motifs from known cardiomyocyte transcription factors (TF). Cardiomyocyte TF motifs (for example, Gata4 and 6, Mef2c, Hand2, Nkx2.5, Tbx5, and Tbx20) were enriched in the fragments highly expressed in the cardiomyocytes treated with the skeletal library. Correspondingly, skeletal TF motifs (for example, Myf5. MyoD, and MyoG) were enriched in the fragments highly expressed in the skeletal myoblasts treated with the cardiac library.

The methods detailed herein identified DNA fragments that may form a promoter that drives gene expression specific for various tissues and cells, such as skeletal myoblasts, cardiomyocytes, satellite cells, and neuronal subtypes, while avoiding expression in other tissues and cell types.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method of identifying a promoter sequence for directing tissue-specific or cell-specific gene expression, the method comprising: identifying one or more DNA fragments from a first cell type, wherein each DNA fragment is 50 nt to 150 nt in length and present in a genomic region comprising at least one epigenetic feature in the first cell type, wherein the epigenetic feature is selected from open chromatin and histone mark and DNA methylation, and wherein the epigenetic feature is not present in the same genomic region in at least one second cell type; inserting into a vector at least one promoter sequence comprising at least one of the one or more DNA fragments and at least one sequence tag, wherein the sequence tag comprises a polynucleotide sequence that is specific for each DNA fragment; transducing one or more vectors into an expression cell; and determining the level of transcription of the sequence tag in the expression cell, wherein an increased level of transcription of the sequence tag in the expression cell relative to a control indicates that the promoter sequence directs tissue-specific or cell-specific gene expression.

Clause 2. The method of clause 1, wherein the vector further includes a polynucleotide encoding a reporter downstream of and operably linked to the at least one promoter sequence.

Clause 3. The method of clause 2, wherein the reporter comprises a fluorescent protein.

Clause 4. The method of any one of clauses 1-3. the method further comprising sequencing the one or more DNA fragments prior to inserting into a vector the at least one promoter sequence.

Clause 5. The method of any one of clauses 1-4, the method further comprising synthesizing the one or more DNA fragments prior to inserting into a vector the at least one promoter sequence.

Clause 6. The method of any one of clauses 1-5, wherein the epigenetic feature is not present in the same genomic region in at least two second cell types.

Clause 7. The method of any one of clauses 1-5, wherein the epigenetic feature is not present in the same genomic region in at least one second cell type but is present in the same genomic region in at least one third cell type.

Clause 8. The method of any one of clauses 1-7, wherein the first cell type and the second cell type are from different tissues.

Clause 9. The method of any one of clauses 1-7. wherein the first cell type and the second cell type are different cell types.

Clause 10. The method of any one of clauses 1-9, wherein the vector comprises a combination of at least about 2, at least about 3, or at least about 4 of the one or more DNA fragments.

Clause 11. The method of any one of clauses 1-9, wherein the promoter sequence comprises a combination of at least about 2, at least about 3, or at least about 4 of the one or more DNA fragments.

Clause 12. The method of any one of clauses 1-11, wherein the one or more DNA fragments are identified by comparing DNAse hypersensitivity data for the first cell type to DNAse hypersensitivity data for the second cell type.

Clause 13. The method of clause 12, wherein the DNAse hypersensitivity data is obtained by DNAse-seq or ATAC-seq.

Clause 14. The method of any one of clauses 1-11, wherein the one or more DNA fragments are identified by comparing histone modification data for the first cell type to histone modification data for the second cell type.

Clause 15. The method of clause 14, wherein the histone modification data is obtained by ChIP-seq.

Clause 16. The method of any one of clauses 1-15, wherein the level of transcription of the sequence tag in the expression cell is determined by quantitative DNA sequencing.

Clause 17. The method of any one of clauses 1-16, the method further comprising comparing the level of transcription of the sequence tag in the expression cell to the level of transcription of a different sequence tag corresponding to a different promoter sequence.

Clause 18. The method of any one of clauses 1-16, the method further comprising comparing the level of transcription of the sequence tag in the expression cell to the level of transcription of the same sequence tag in a different cell type.

Clause 19. The method of any one of clauses 1-18, wherein the vector is a lentiviral or adeno-associated viral (AAV) vector.

Clause 20. The method of any one of clauses 1-19, wherein the first cell type is a cardiac cell, skeletal muscle cell, smooth muscle cell, endothelial cell, intestinal cell, epithelial cell, liver cell, retinal cell, hematopoietic stem cell, satellite cell, CNS cell, astrocyte, glial cell, brain cell, neuronal cell, or neuronal subtype cell.

Clause 21. The method of clause 20, wherein the neuronal subtype cell is a dopaminergic neuron, gabaergic neuron, or glutamatergic neuron.

Clause 22. A vector comprising the tissue-specific or cell-specific promoter sequence identified by the method of any one of clauses 1-21.

Clause 23. The vector of clause 22, wherein the tissue-specific or cell-specific promoter sequence is a cardiac muscle-specific promoter or a skeletal muscle-specific promoter.

1. A method of identifying a promoter sequence for directing tissue-specific or cell-specific gene expression, the method comprising:
identifying one or more DNA fragments from a first cell type, wherein each DNA fragment is 50 nt to 200 nt in length and present in a genomic region comprising at least one epigenetic feature in the first cell type, wherein the epigenetic feature is selected from open chromatin and histone mark and DNA methylation, and wherein the epigenetic feature is not present in the same genomic region in at least one second cell type;
inserting into a vector at least one promoter sequence comprising at least one of the one or more DNA fragments and at least one sequence tag, wherein the sequence tag comprises a polynucleotide sequence that is specific for each DNA fragment;
transducing one or more vectors into an expression cell; and
determining the level of transcription of the sequence tag in the expression cell, wherein an increased level of transcription of the sequence tag in the expression cell relative to a control indicates that the promoter sequence directs tissue-specific or cell-specific gene expression.

2. The method of claim 1, wherein the vector further comprises a polynucleotide encoding a reporter downstream of and operably linked to the at least one promoter sequence.

3. The method of claim 2, wherein the reporter comprises a fluorescent protein.

4. The method of claim 1, the method further comprising sequencing the one or more DNA fragments prior to inserting into a vector the at least one promoter sequence.

5. The method of claim 1, the method further comprising synthesizing the one or more DNA fragments prior to inserting into a vector the at least one promoter sequence.

6. The method of claim 1, wherein the epigenetic feature is not present in the same genomic region in at least two second cell types.

7. The method of claim 1, wherein the epigenetic feature is not present in the same genomic region in at least one second cell type but is present in the same genomic region in at least one third cell type.

8. The method of claim 1, wherein the first cell type and the second cell type are from different tissues.

9. The method of claim 1, wherein the first cell type and the second cell type are different cell types.

10. The method of claim 1, wherein the vector comprises a combination of at least about 2, at least about 3, or at least about 4 of the one or more DNA fragments.

11. The method of claim 1, wherein the promoter sequence comprises a combination of at least about 2, at least about 3, or at least about 4 of the one or more DNA fragments.

12. The method of claim 1, wherein the one or more DNA fragments are identified by comparing DNAse hypersensitivity data for the first cell type to DNAse hypersensitivity data for the second cell type.

13. The method of claim 12, wherein the DNAse hypersensitivity data is obtained by DNAse-seq or ATAC-seq.

14. The method of claim 1, wherein the one or more DNA fragments are identified by comparing histone modification data for the first cell type to histone modification data for the second cell type.

15. The method of claim 14, wherein the histone modification data is obtained by ChIP-seq.

16. The method of claim 1, wherein the level of transcription of the sequence tag in the expression cell is determined by quantitative DNA sequencing.

17. The method of claim 1, the method further comprising comparing the level of transcription of the sequence tag in the expression cell to the level of transcription of a different sequence tag corresponding to a different promoter sequence.

18. The method of claim 1, the method further comprising comparing the level of transcription of the sequence tag in the expression cell to the level of transcription of the same sequence tag in a different cell type.

19. The method of claim 1, wherein the vector is a lentiviral or adeno-associated viral (AAV) vector.

20. The method of claim 1, wherein the first cell type is a cardiac cell, skeletal muscle cell, smooth muscle cell, endothelial cell, intestinal cell, epithelial cell, liver cell, parenchymal cell, hepatocyte, adipocyte, fibroblastic cell, Kupffer cell, stromal cell, retinal cell, hematopoietic stem cell, satellite cell, CNS cell, astrocyte, glial cell, brain cell, neuronal cell, or neuronal subtype cell.

21. The method of claim 20, wherein the neuronal subtype cell is a dopaminergic neuron, gabaergic neuron, or glutamatergic neuron.
